(19) United States
(12) Patent Application Publication
(10) Pub. No.: US 2013/0116224 A1
(43) Pub. Date: May 9, 2013

(54) Method and Composition for Treatment of Tumors

(71) Applicant: David Gershon, New York, NY (US)

(72) Inventor: David Gershon, New York, NY (US)

(21) Appl. No.: 13/808,793

(22) PCT Filed: Jul. 8, 2011
(86) PCT No.: PCT/US11/43467

§ 371 Date: Jan. 7, 2013

Related U.S. Application Data
(60) Provisional application No. 61/505,831, filed on Jul. 8, 2011.
(60) Provisional application No. 61/362,576, filed on Jul. 8, 2010.

(51) Int. Cl. A61K 31/555

(52) U.S. Cl. CPC A61K 31/555
USPC 514/184

(57) ABSTRACT

A method for ameliorating tumors which result from high or increased activity of Gli by treating a human subject evidencing tumor growth by treating the subject with a deactivating effective amount of CTC-96 to reduce or inhibit tumor growth.

### BACKGROUND OF THE INVENTION

CTC-96 (Doxovir), is a cobalt III Schiff base complex with a molecular weight of 550. In its structure it resembles the core of cobalamine (Vitamin B12). Originally, it was synthesized as a superoxide scavenger to serve as a non-steroidal anti-inflammatory agent (1)¹. CTC-96 (Doxovir™) is a small cobalt (III) Schiff base complex Its structure and method of preparation are disclosed in U.S. Pat. No. 5,756,491, the entire contents of which are hereby incorporated by reference. The compound's mode of action is based on its binding to the nitrogen of the imidazole ring of some histidine residues in proteins (1). It can thus disrupt the structure and function of Zn fingers that contain one or two histidines coordinated about a zinc ion in proteins (2). It can also disrupt other Zn containing motifs in proteins. Moreover, it inhibits enzymes that contain histidine in their active site such as thermolysin and thrombin (3) and is, therefore, expected to effectively inhibit some other serine and cysteine proteases. ¹ numbers in parentheses refer to the numbered references at the end of this document

More specifically, biochemical studies in vitro and in vivo revealed it to bind specifically to the nitrogen of the imidazole of selected histidines (2). Due to the binding of histidine, it was found that this cobalt Schiff base molecule selectively disrupts the structure and function and inactivates some proteases with essential histidines and their active site (2). It also disrupts the structure of zinc fingers in transcription factors such as, for instance, SP1. CTC-96 binds to the Zn-coordinating histidines which lead to the displacement of the zinc ion from the zinc finger (3).

The Hedgehog (HH) signaling system and pathway are vitally involved in animal development and is essential in the regulation of cell fate and number in the brain and other organs and is essential for stem cell maintenance (reviewed in 4 and 5). Malfunction of HH signaling contributes to malignancy in several types of cancer such as glioblastoma and medulloblastoma in the brain, multiple myeloma, myeloid leukemia, melanoma, basal cell carcinoma colon, pancreatic and prostate cancers. The malignant state of these tumors is, at least, partially due to up-regulation of the Gli genes which encode particular transcription factors (4,5).

These genes are located downstream in the gene activation cascade of the HH signal pathway; they encode Gli transcription factors which activate the ultimate HH target genes responsible for the malignant transformation of cells (see FIG. 2 in 4 and FIG. 2 in 5). The HH-Gli signaling regulates, for instance, human glioma growth, cancer stem cell self-renewal and tumorigenicity in gliomas (6) in colon cancer (7), pancreatic cancer (7a) BCC (7b) and melanoma (7c). In the latter, HH is implicated in stem cell maintenance and self renewal, stromal pathway relevant to metastasis and, in general, malignant transformation (7a).

There is evidence that a substantial proportion of glioblastomas are associated with up-regulation of Gli genes (8). Quin et al showed high correlation between Sonic HH-Gli and PTEN expression in tumor cell proliferation that is associated with reduced survival time (9). Also there is evidence that a Gli1 splice variant promotes glioblastoma cell migration and invasion (10). In addition, Becher et al, showed that the level of Gli activity is correlated with tumor grade in gliomas induced in mice (11).

It is noted that Gli activation can occur in an HH-independent, non-canonical fashion, by several signaling pathways such as RAS and AKT (FIG. 2 in 4; 12, FIG. 2). It is thus a preferred target for therapy rather than upstream genes that belong to the HH pathway.

### SUMMARY OF THE INVENTION

The present invention resides in the method of treatment of a human subject with a tumor mitigating effective amount of CTC-96. In particular, it resides in a method for ameliorating tumors, which result from high or increased activity of Gli by treating a human subject, evidencing tumor growth with deactivating effective amount of CTC-96 2 reduce or inhibit tumor growth.

### BRIEF DESCRIPTION OF THE DRAWINGS

FIG. 1A1-1D4 are line graphs depicting strain growth for various different cell lines at various density seeding rates.

FIG. 2A-2D are bar graphs depicting strain growth for various different cell lines at various density seeding rates.

FIG. 3A-3D are line graphs depicting cell proliferation for various cell lines at increasing seeding densities.

FIG. 4A1-4D4 are line graphs depicting cell growth of several cell lines with period of treatment with CTC-96 . . . .

FIG. 5A-5d are bar graphs depicting cell growth of several cell lines with period of treatment with CTC-96.

FIG. 6A1-6AD are line graphs depicting cell growth of several cell lines with period of treatment with CTC-96.

FIG. 7A-7D are bar graphs depicting cell line growth for various cell lines at different seeding densities.

### DETAILED DESCRIPTION OF THE INVENTION

A) Gli transcription factors are highly implicated in several aspects of tumorigenicity of a variety of cancers. They bind to Gli binding sites on DNA in the promoter region of target genes. They are involved in the activation of many genes that are necessary for the malignant transformation of a variety of cell types that form specific tumors. Gli 1-3 are therefore, a target for specific inhibitory drugs interacting directly with these protein resulting in Zn ejection and thus loss of the ability to bind DNA (e.g., 3). CTC-96 is a very likely candidate for such specific inhibition of Gli activity. This is based on the fact that the Gli proteins have five tandem Zn fingers with high concentration of histidines in the linker sequences between the Zn fingers. This number of Zn fingers is unusually high and with the abundance of histidines in the linkers of these Zn fingers in Gli molecule makes Gli a prime target for CTC 96. Thus, a tumor cell with many active Gli molecules which are responsible for its proliferation, de-differentiation and invasiveness (and perhaps its viability), is sensitive and susceptible to relatively very low concentrations of CTC-96 compared to normal cells. Gli activity is low in quiescent cells in the adult brain and most other organs including the majority of normal tissue-specific stem cells. CTC-96 acts relatively safely as a tumor inhibitor, by virtue of its anti-Gli activity. It can also affect the viability of the tumor cells by inducing apoptosis.

B) In addition, at least in the early stages of invasion of tumors the interaction with ECM involves some MMPS (containing Zn as a structural element) and serine proteases which have a critical histidine in their active site, they constitute another likely target for CTC-96, which also retards the early events of invasion and metastasis.

C) Moreover, it is a well known fact that since inflammation plays a role in the development of tumors. CTC-96 which exhibits anti-inflammatory properties can helps retard the maturation of various tumors.

D) Considering brain tumors, CTC-96 will transcend the blood brain barrier since it is close in structure to the core of cobalamine. Cobalamine enters the brain and contributes to myelin formation. It is also found in the CSF after oral administration.

### EXPERIMENTAL

First Stage:

Determination of the Viability and Rate of Proliferation of Drug-Treated Tumor and Normal Cells.

Proliferation is studied by BUDR incorporation and cell counts. Viability is determined by relevant staining and cell counting. Subsequently IC₅₀ is determined and comparison of the reaction of the tumor and the normal cells is monitored. The effect of the drug on cell motility is determined where relevant. A factor of 5-10 or better between the effect of the drug on tumor cells and normal cells in any of the parameters of proliferation, invasiveness and viability cells is considered a success.

Second Stage:

The effect of the drug on xenografts of human of relevant Gli-dependent tumors in the appropriate mouse strains (Immune compromised or existing specially constructed strains, e,g., for pancreatic cancer, (13) and melanoma, (7c) are tested. The growth and invasiveness of the tumors in mouse brains and other organs are evaluated in mice IV injected with the drug twice daily at doses of vehicle only or 0.1, 0.25, 1.0 and 5.0 mg/kg. The doses are based on safety determinations carried out in preparation for FDA approval.

Accordingly, the invention comprises a novel system of direct treatment of Gli which is applicable to several major cancers ranging from brain tumors (glioblastomas and medulloblastomas), colon, pancreatic, prostate cancers, BCC and melanomas etc. This drug acts directly on Gli transcription factors which are over-expressed in the tumors regardless of whether they are activated by the hedgehog pathway or by other non-canonical oncogenic pathways. It can be used to treat a broad spectrum of tumors.

The invention also comprises a composition which contains an antitumor effective amount of CTC-96. The inventive composition may also contain a pharmaceutically acceptable carrier admixed with the CTC 96. The inventive composition may be administered by a variety of well-established medicinal routes including intravenously, intraperitoneally, intramuscularly, orally or intranasally.

The following example sets forth an evaluation of the preferential proliferation inhibition and killing of targeted human malignant cell lines, which possess Gli-1 up-regulation by CTC-96.

### Example

### A. Cell Line Selection

Nine cell lines were obtained from ATCC as listed in Table 1. Healthy cultures were established by maintaining/propagating cells using ATCC recommended medium and conditions. These are listed in Table I. All cell lines grew at an acceptable rate, except for MDA-MB-231, which divided too slowly to be assayed.

TABLE 1

| | ATCC No. | Alternative Name | Origin/Description |
|---|---|---|---|
| 1 | HTB-186 | Daoy | Desmoplastic cerebellar |
| | | | mediulloblastoma |
| 2 | HTB-26 | MDA-MB-231 | (breast) mammary gland, |
| | | | adenocarcinoma, epthelia |
| 3 | CRL-1740 | LNCaP clone FGC | Prostate, derived from |
| | | | metastatic site, left, |
| | | | supraclavicular lymph node |
| 4 | CRL-11609 | RWPE-1 | Prostate, epithelial (has HPV |
| | | | 18 sequences: BSL 2) |
| 5 | HTB-64 | Malme-3M | Malignant melanoma derived |
| | | | from metastatic site (lung) |
| 6 | HTB-102 | Malme-3 | Skin, fibroblast normal |
| 7 | CRL-1682 | AsPC-1 | Pancreas, adenocarcinoma, |
| | | | metastatic |
| 8 | CCL-75 | WI 38 | Female diploid lung cells, |
| | | | embryonic |
| 9 | CCL-186 | IMR 90 | Male diploid lung cells, |
| | | | embryonic |

After cells went through three passages. whole cell lysates were prepared from the 8 cell lines. Gli-1 expression was evaluated using Western blot. Three cell lines slowed up-regulation of Gli-1. They were LNCaP, Malme-3 and Malme-3M. There was no detectable expression of Gli-1 in the other 5 cell lines. See Table 2.

TABLE 2

| | | Alternative | |
|---|---|---|---|
| | ATCC No. | Name | Origin/Description |
| 1 | HTB-186 | Daoy | − |
| 2 | HTB-26 | MDA-MB-231 | ? |
| 3 | CRL-1740 | LNCaP clone | + |
| | | FGC | |
| 4 | CRL-11609 | RWPE-1 | − |
| 5 | HTB-64 | Malme-3M | + |
| 6 | HTB-102 | Malme-3 | + |
| 7 | CRL-1682 | AsPC-1 | − |
| 8 | CCL-75 | WI 38 | − |
| 9 | CCL-186 | IMR 90 | − |

LNCaP and its sister normal cell line RWPE were selected as one pair for further study. Malme-3 was supposed to be a normal cell line and up-regulation of Gli-I was unexpected. To facilitate the study, Malme-3M was paired with IMR90 for further study.

### B. In Vitro Anti-Cancer Efficacy Study of CTC-96 in Cell Lines Maintained in ATCC Recommended Growth Condition Using Approved Protocol GP687

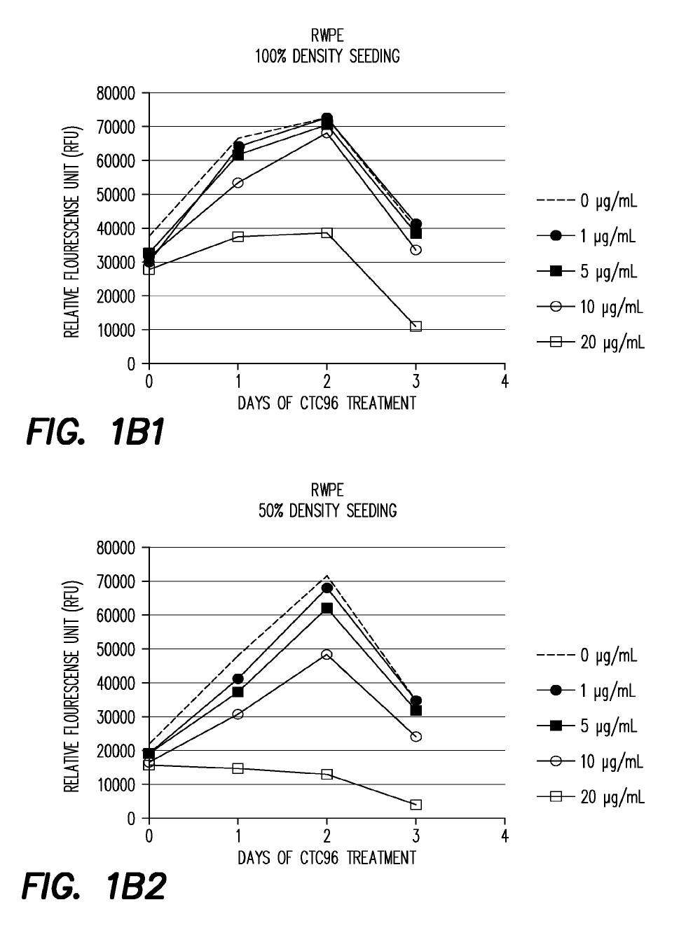

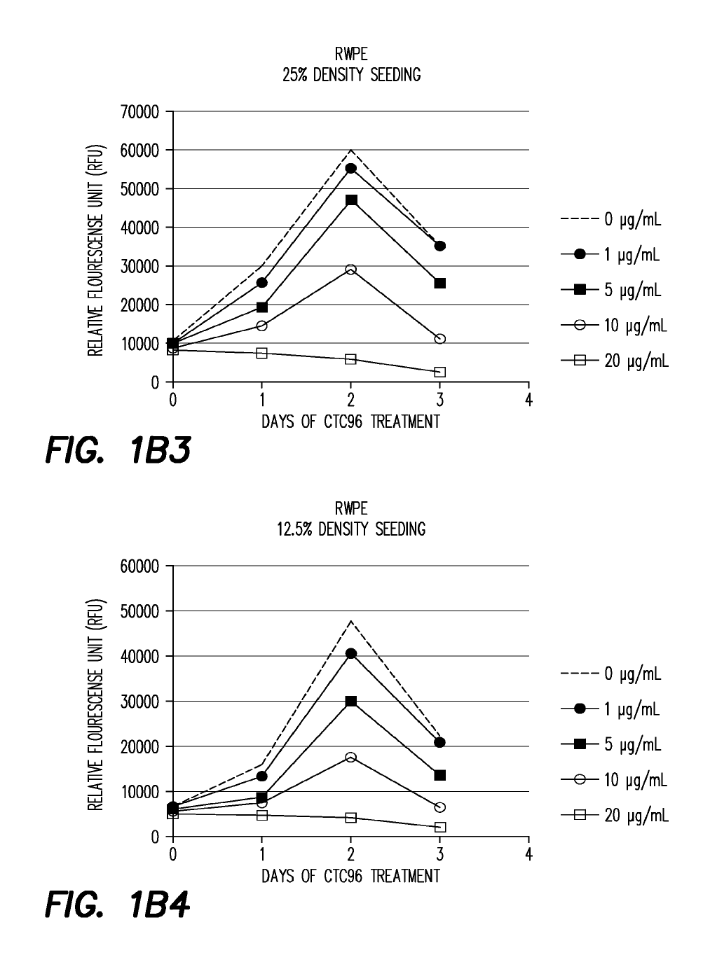

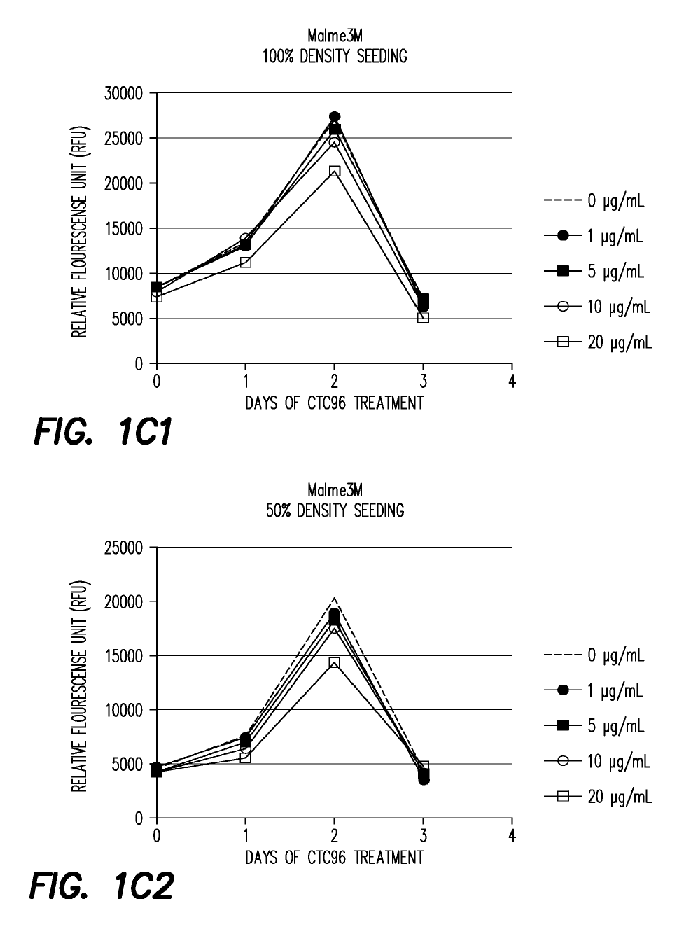

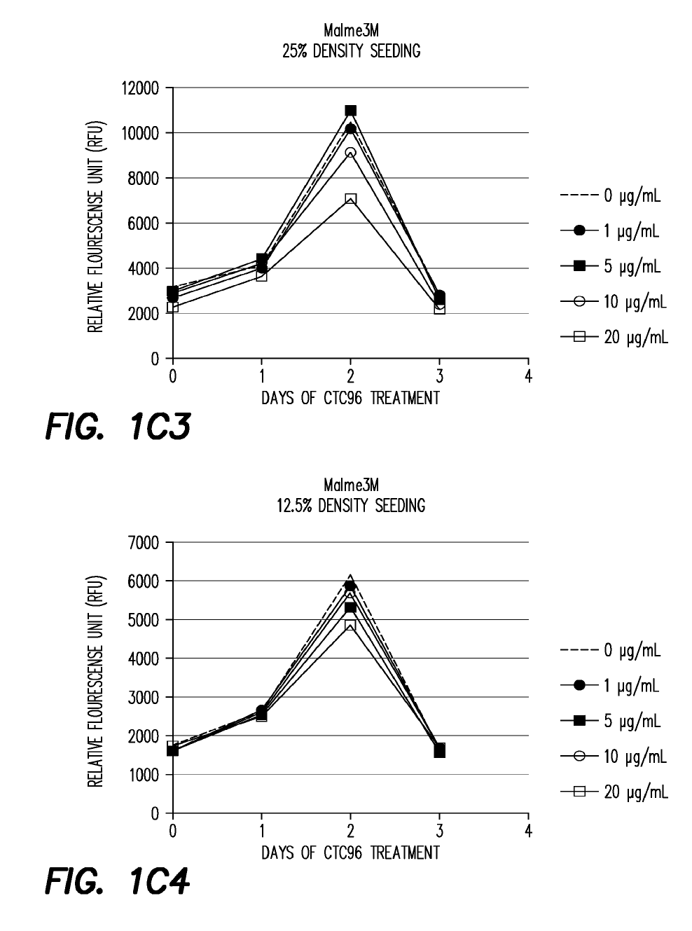

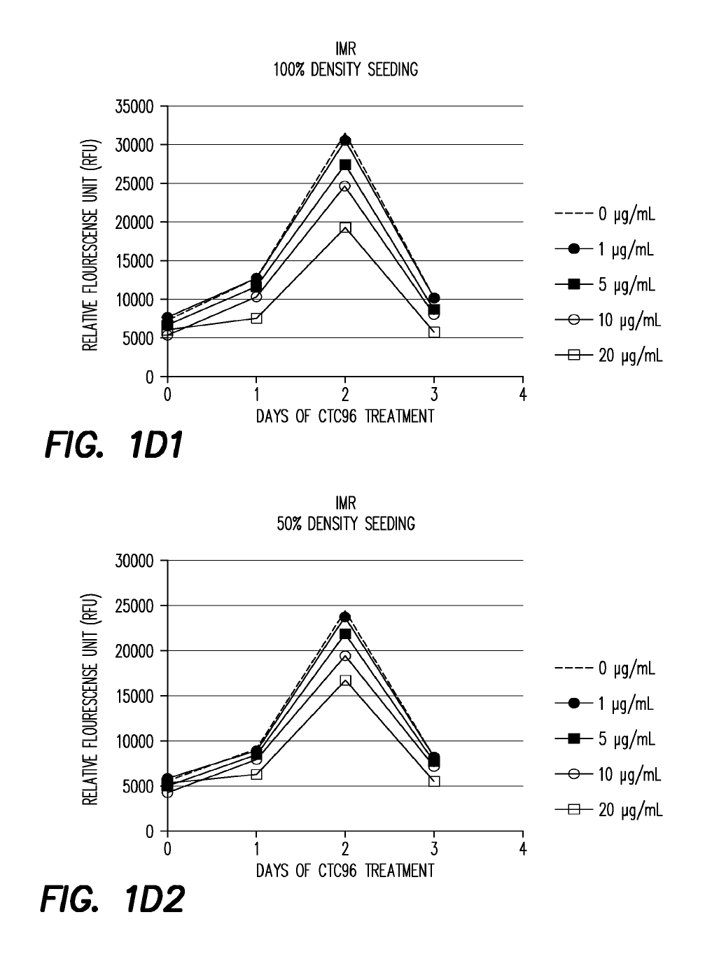

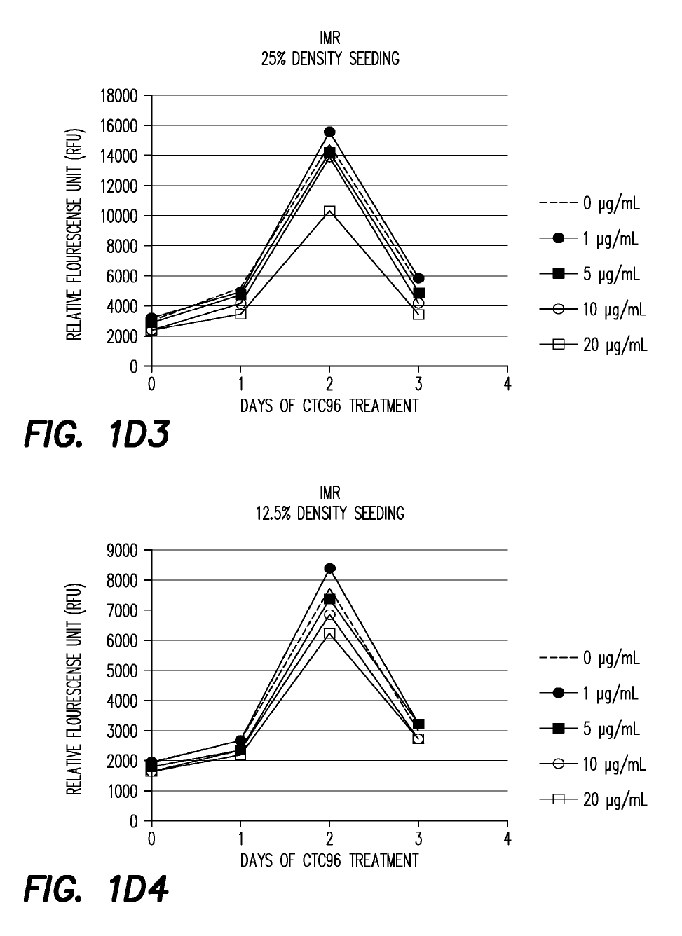

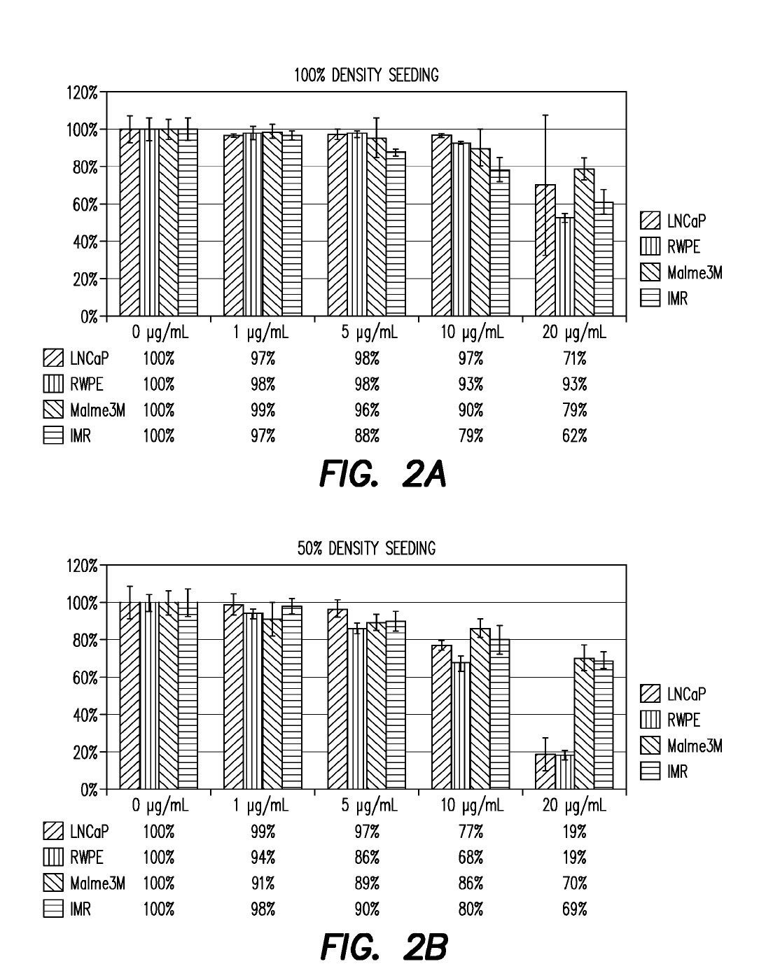

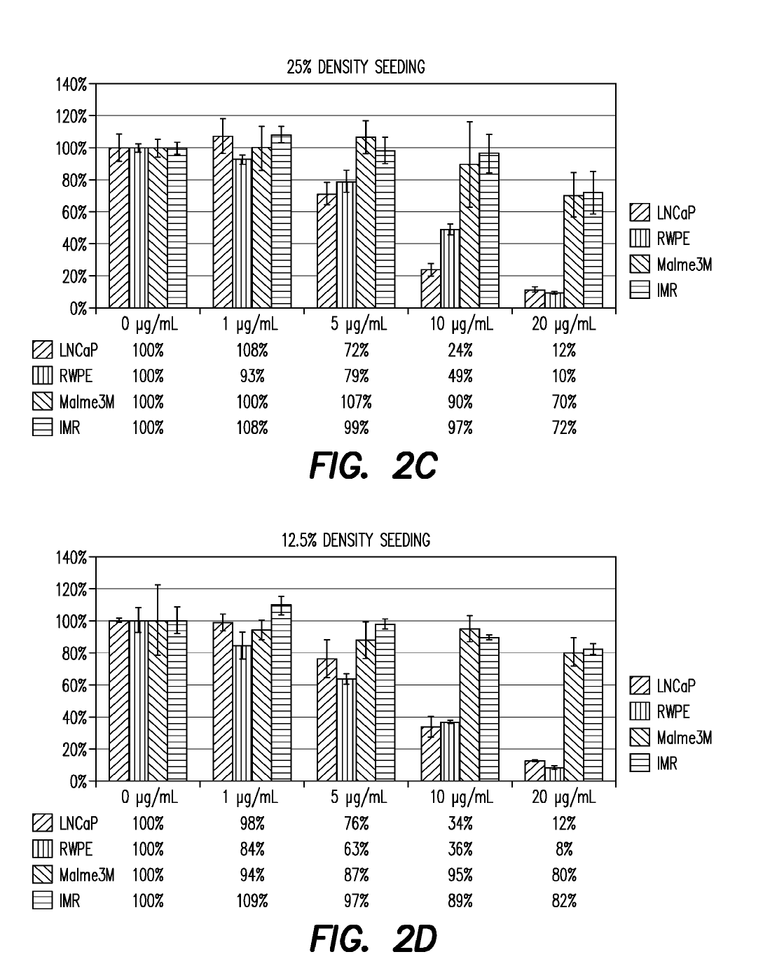

Generally speaking, all four cell lines showed a dose-dependent response to treatment with CTC-96. However, LNCaP and RWPE cells demonstrated more growth inhibition in response to CTC-96 treatment than did Malme-3M and IMR cells as shown in FIG. 2. For example in FIG. 2C, on day 2 with 20 μg/mL CTC-96. LNCaP and RWPE only exhibited 12% and 10% of the total cells in comparison to untreated controls, while, Malme-3M and IMR still maintained 70% and 72%. This difference among the cell lines was quite consistent at all initial seeding densities except for the highest (100%). At the three lower density seedings, the decrease in relative fluorescence units (RFU) was in a near linear relationship with increase of CTC-96 dose for LNCaP and RWPE cells. Both cell types began to show a response to CTC-96 at 5 μg/mL. (see FIGS. 2 C and D).

Low cell density seedings revealed better response to CTC-96 treatment.

### C. Interpretation

The most critical variables which may affect CTC-96 efficacy in the study were: serum concentration and base medium composition. Of the latter the concentration of histidine is particularly consequential. Therefore, one base medium, RPM11640, was chosen for testing of all cell lines in the ensuing studies in order to eliminate any interference of the varied histidine levels in the test system and serum concentration was standardized to lower levels to minimize interference with CTC-96 activity while allowing all cell lines to proliferate.

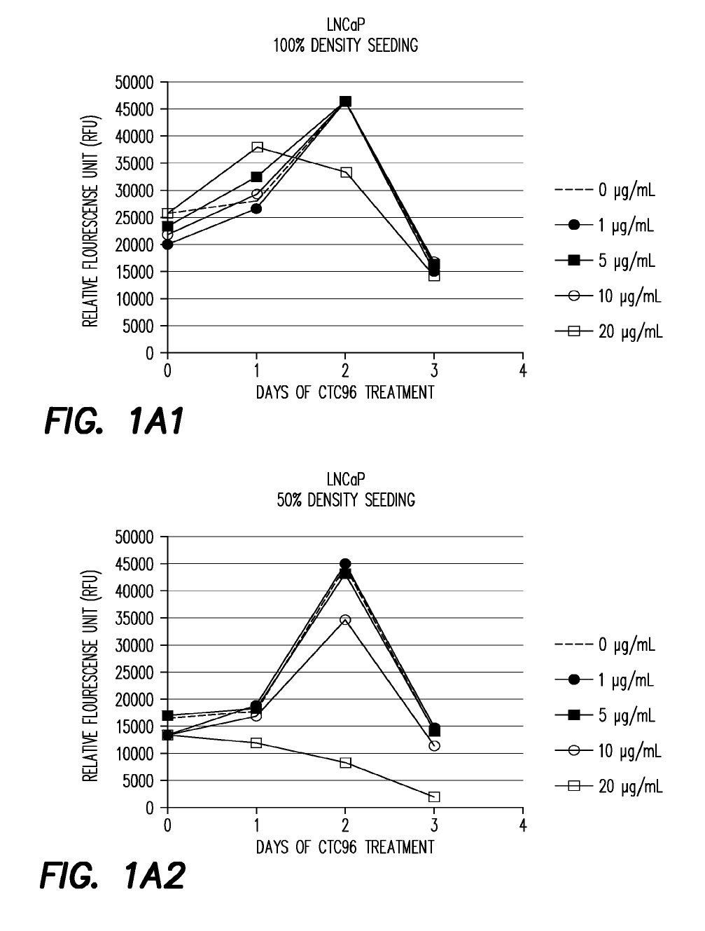

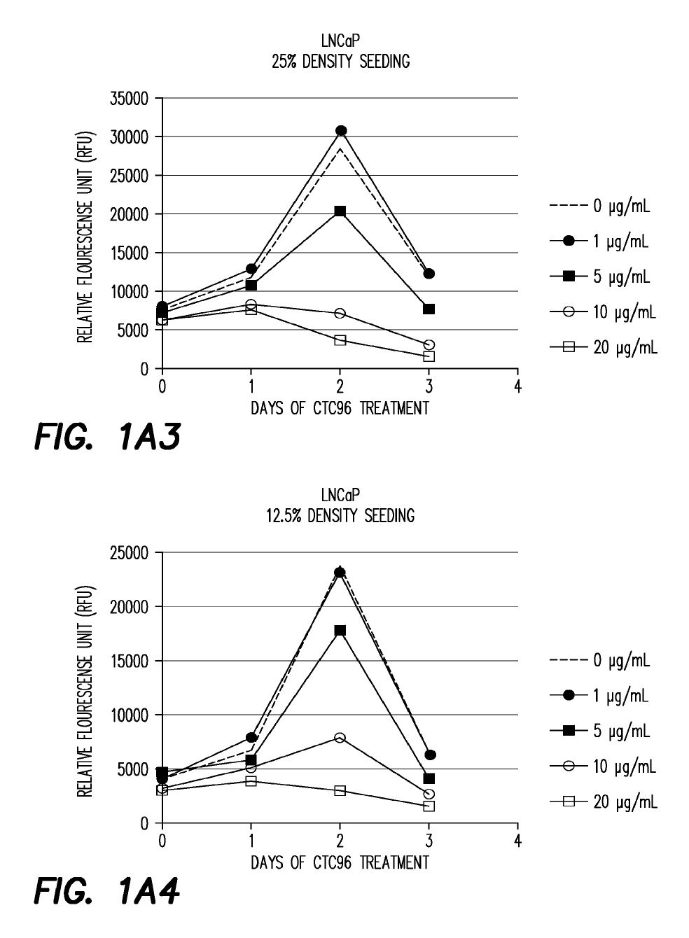

When cell proliferation data were obtained for all time points, the RFU reading on day 3 was considerably lower than the projected trend line as shown in FIG. 1. The possible cause was the variation in the incubation time as described in GP687 9.5.2.3. The cell viability and proliferation assay manual does not specify incubation time. Due to time constraints, the incubation time carried out was shorter on day three than for the previous three time points. The longer the incubation time, the greater the RFLI measured. Therefore, day 3 incubation data were not included in the analysis, see FIG. 2. In the ensuing studies, incubation time was standardized to 2 hours in order to allow RFU to become comparable for all time points. Different cell density seedings allowed this study to maximally evaluate the anticancer efficacy of CTC-96. In vitro anticancer effectiveness can be categorized into: killing and inhibiting effects. High density seeding provides maximal numbers of cells to reveal the killing effect, which leads to extensive cell death with quick onset. Also high cell density sometimes provides some protection against cytotoxic effects. Low density seeding reduces the effects of contact inhibition in non-malignant cell lines and allows longer incubation with the drug. The longer treatment time allows observation of maximum inhibitory drug effects. In addition, low density cell seeding allows untreated cells to undergo several cell divisions.

In this study, four seeding densities of 100%, 50%, 25% and 12.5% were planned for each cell line. However, the actual seeding conditions was about 50% confluency was achieved for the 100% planned density (LNCaP and RWPE). and it was roughly 30% for Malme-3M and IMR. The cells in the 50% 25% and 12.5% density seedings were evenly spaced, and exhibited lower cell density proportionally.

The killing effect was not significantly observed in this phase. Only LNCaP and RWPE with 20 μg/mL CTC-96 had a lower number of cell on day 2 than on day 0 due to net cell loss (See FIG. 1, A2-4 snf B2-4). This was possible due to the antagonistic effect of serum and histidine in the growth medium. Proliferation inhibition was observed ofr LNCaP and RWPE cells seeded at low density (see FIGS. 1 and 2). Based on the RFU reading, untreated cells doubled roughly two rounds in two days, but treated cells proliferated more slowly as a function of the increase in CTC-96 dose. In the ensuing studies, this density seeding. design was routinely utilized.

### D. Acclimation of Candidate Cell Lines in Standardized Low-Serum RPM11640 Medium

“Standardization of the seeding and growth condition for the cell lines in CTC-96 (Method: GP691) anti-cancer efficacy screening”

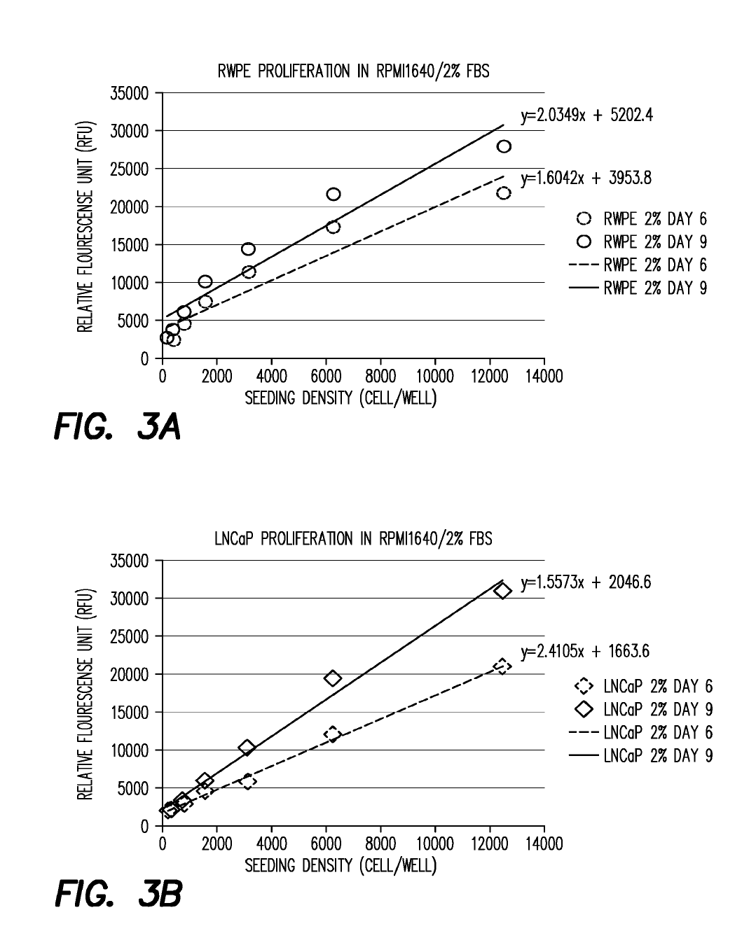

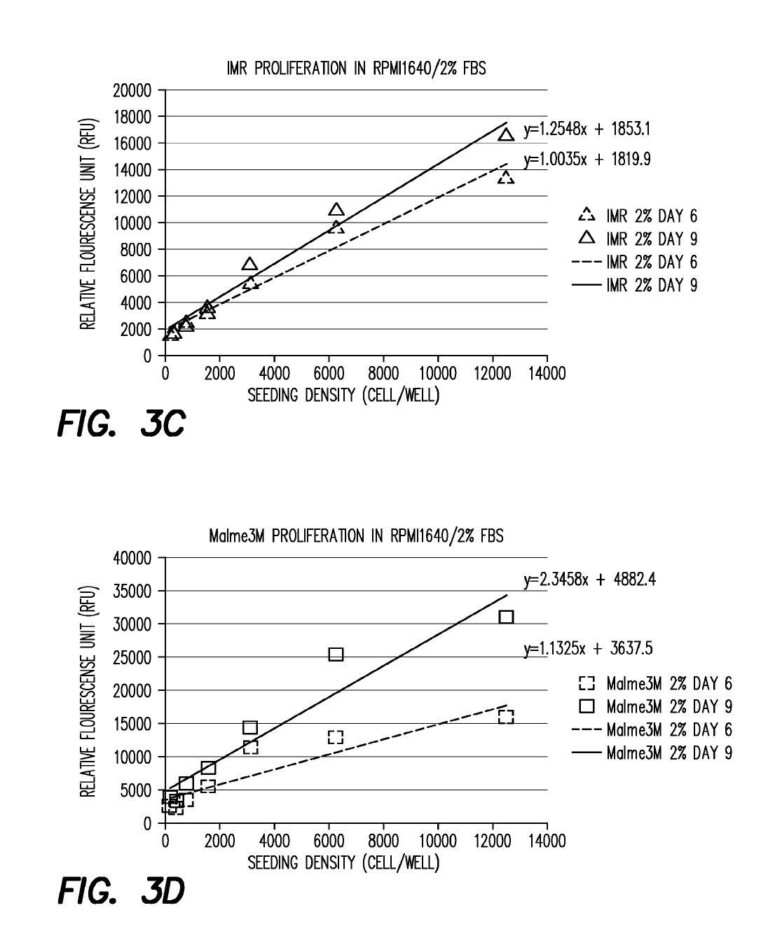

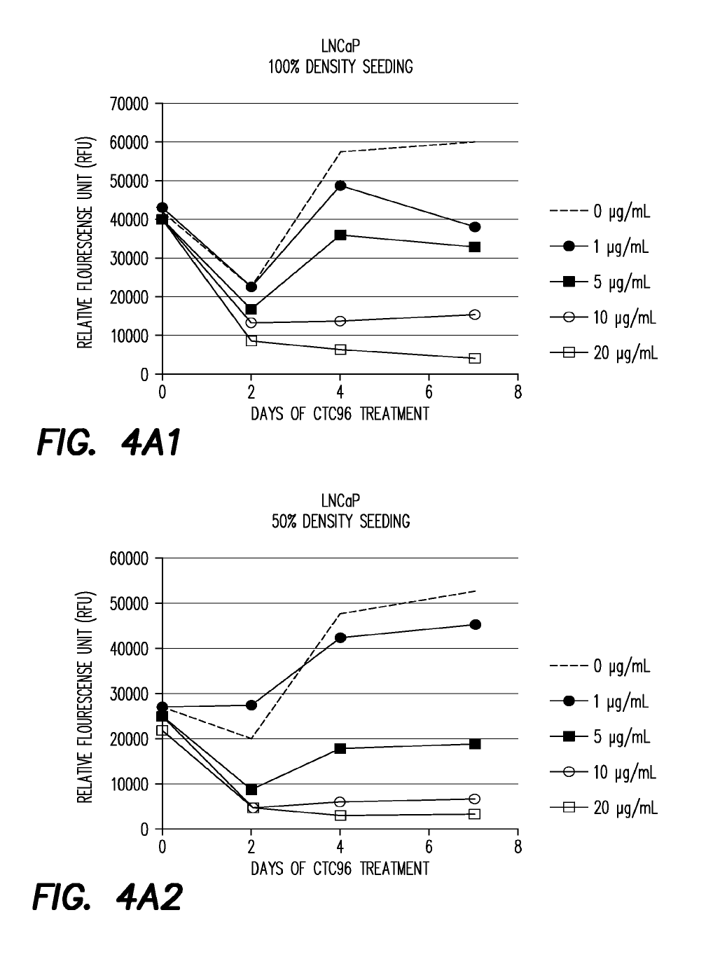

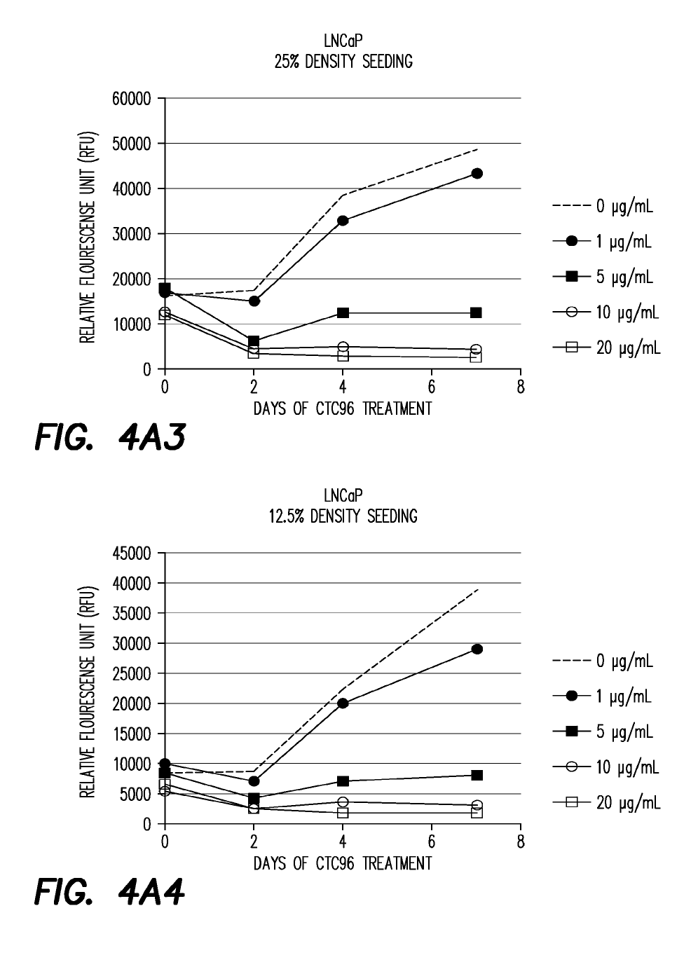

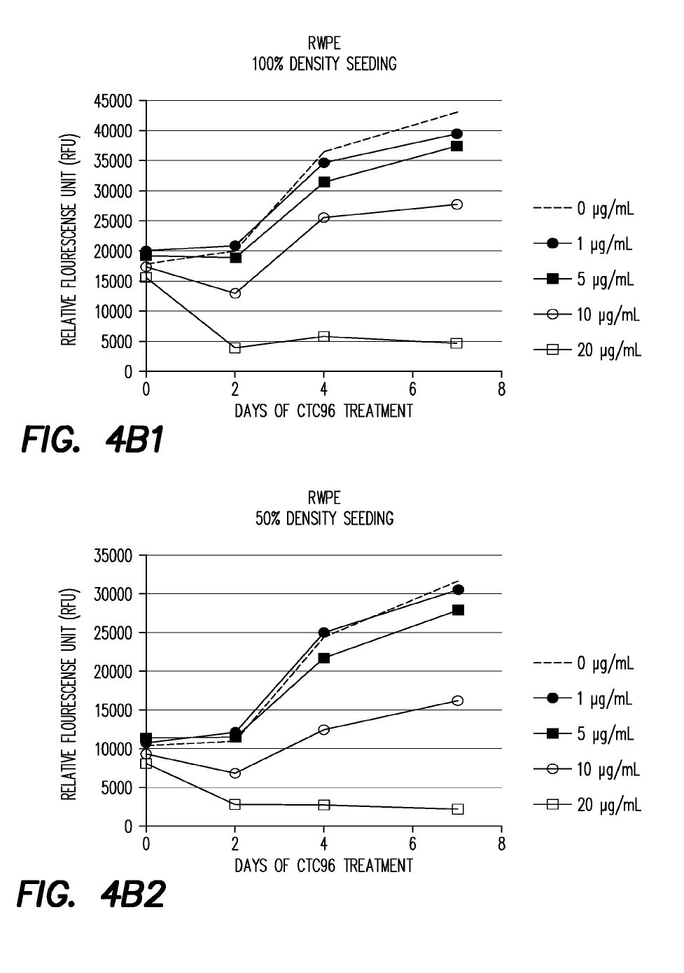

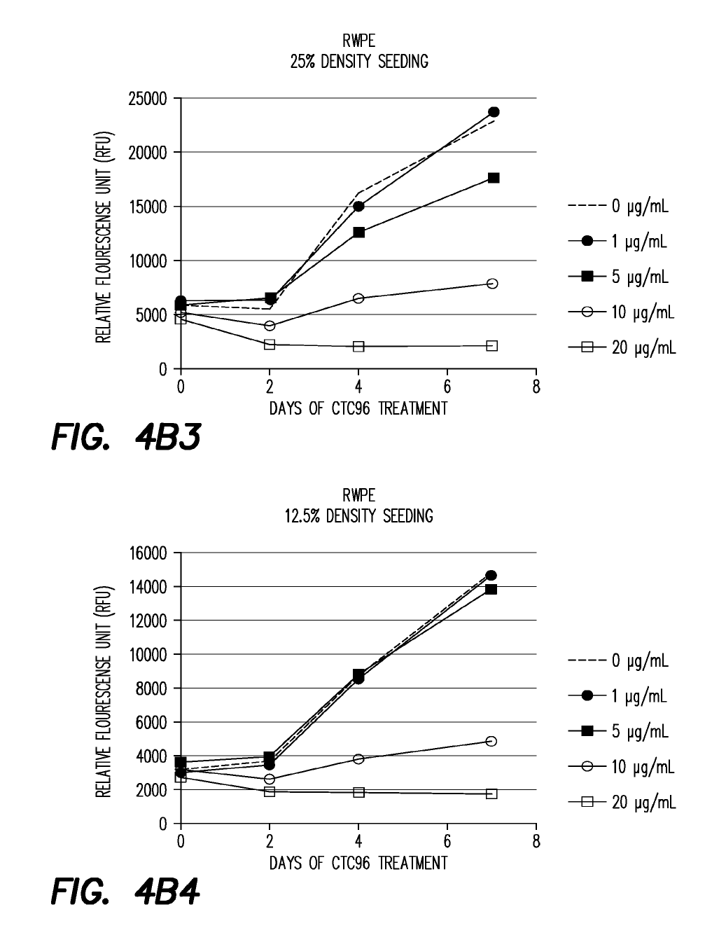

Cell growth and proliferation for all four cell lines was evaluated in RPM1 1640 with 2% or 5% FBS. Both serum levels supported adequate cell growth and proliferation. FIG. 3 shows the results for the RPMII64O/2% FBS condition. Each cell line was seeded at densities ranging from 2×10⁴ cells/well to 40 cells/well using successive 10 to 20 fold dilutions. After 9 days of incubation, some of the high density seedings reached confluency. FIG. 3 depicts RFU readings of day 6 and day 9 of selected non-confluent cell cultures. Based on the trendlines drawn for each day, a linear relationship between RFU reading and cell seeding densitu per well was seen. This indicates that the assay was very sensitive in discriminating viable cell counts in each well. The trendlines of day 9 were above the ones of day 6, showing increases in actual cell number during the additional 3 days. The slope increased from day 6 to day 9. which indicates that cells proliferated faster at relatively higher densities than at lower ones. Thus overall, RPMI1640/2% FBS was found suitable for the following round of CTC-96 treatment study.

All cell lines were acclimated to RPM11640/2% FBS about two weeks ahead of CTC-96 efficacy screening.

### E. Treatment with CTC-96

Treatment was increased from 3 to 7 days. In RPMI1640/2% FBS, the doubling time was 3-4 days for each cell line. Over a 7-day period, untreated control cells doubled about twice (4 fold cell number). The original seeding conditions were kept. 100%. 50%, 25% and 12.5%, for the next CTC-96 treatment. The 25% or 12.5% seedings in the untreated controls were expected to yield a nearly confluent monolayer by the end of 7-day period.

This was considered optimal for revealing proliferation inhibition in treated cells.

### F. Western Blot Study

Western blot confirmed that Gli-1 expression profile did not change in test cell lines after acclimation. Western blots were performed to ensure that adaptation to low-serum growth media had not affected Gli-1 expression in the test cell lines. G.

### G. CTC-96 Exhibited Anti-Cancer Efficacy Against LNCaP and Malme-3M

The study was performed as described in GP689 with modifications as follows:

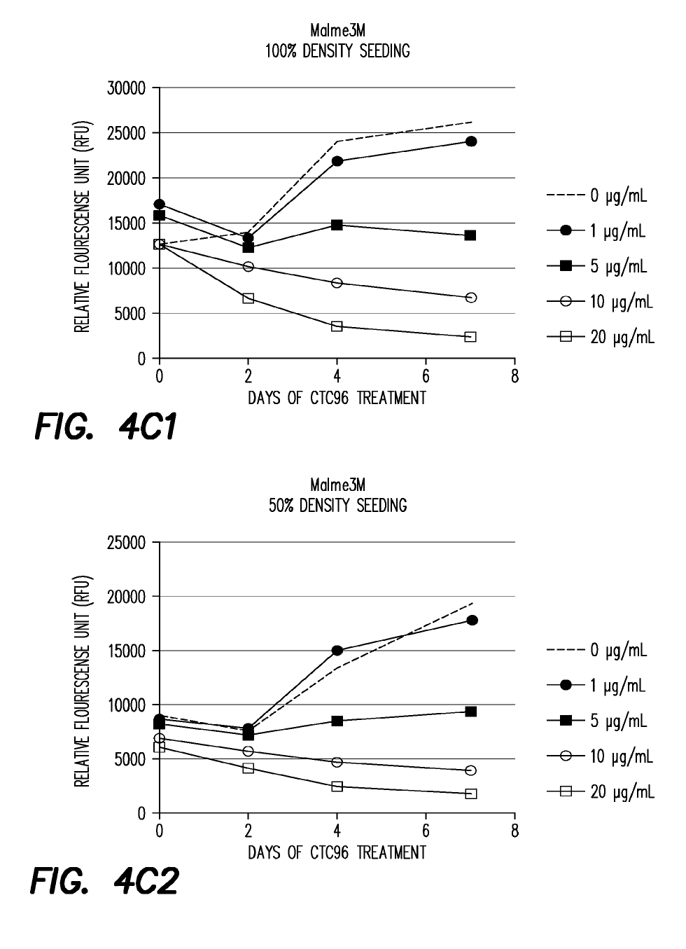

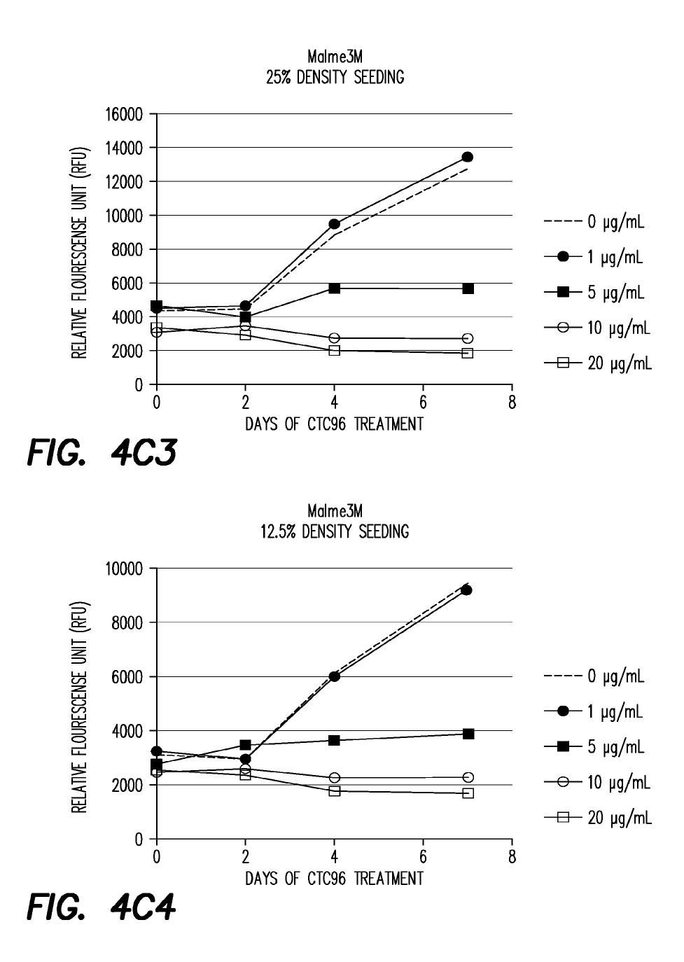

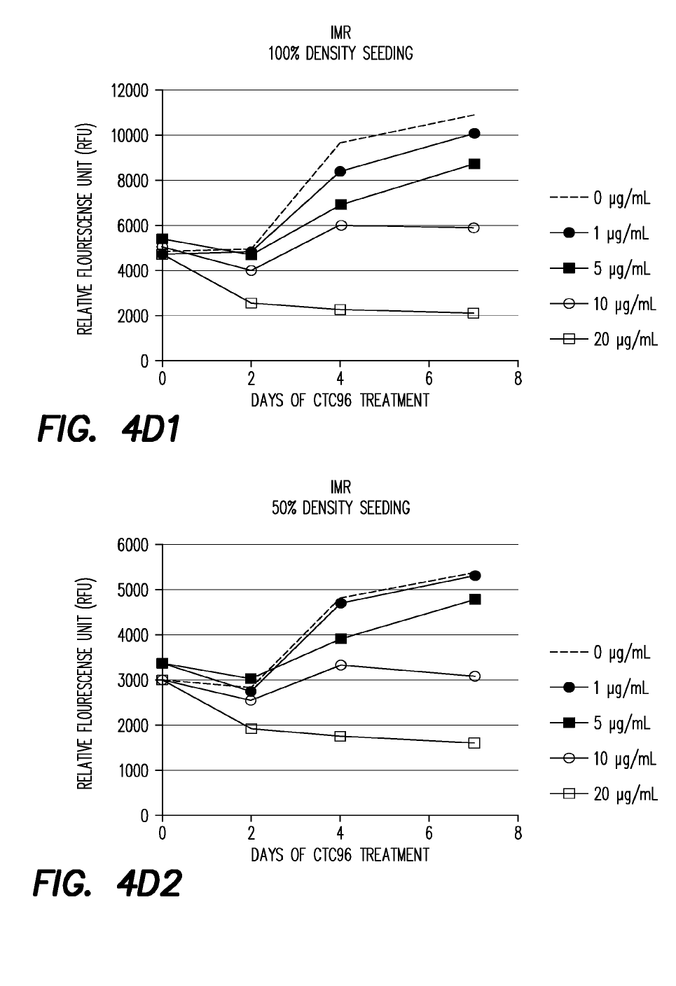

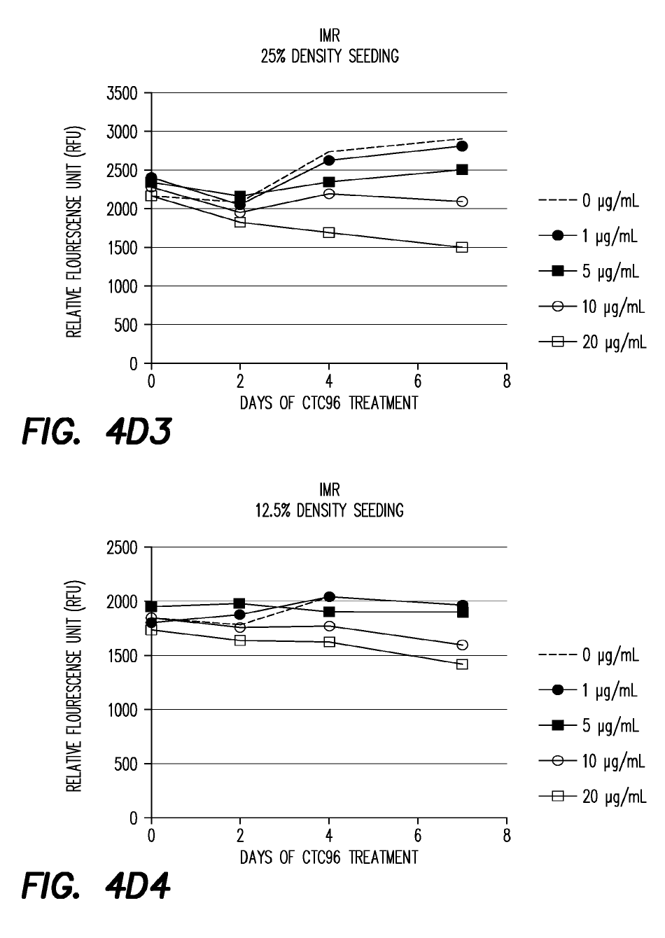

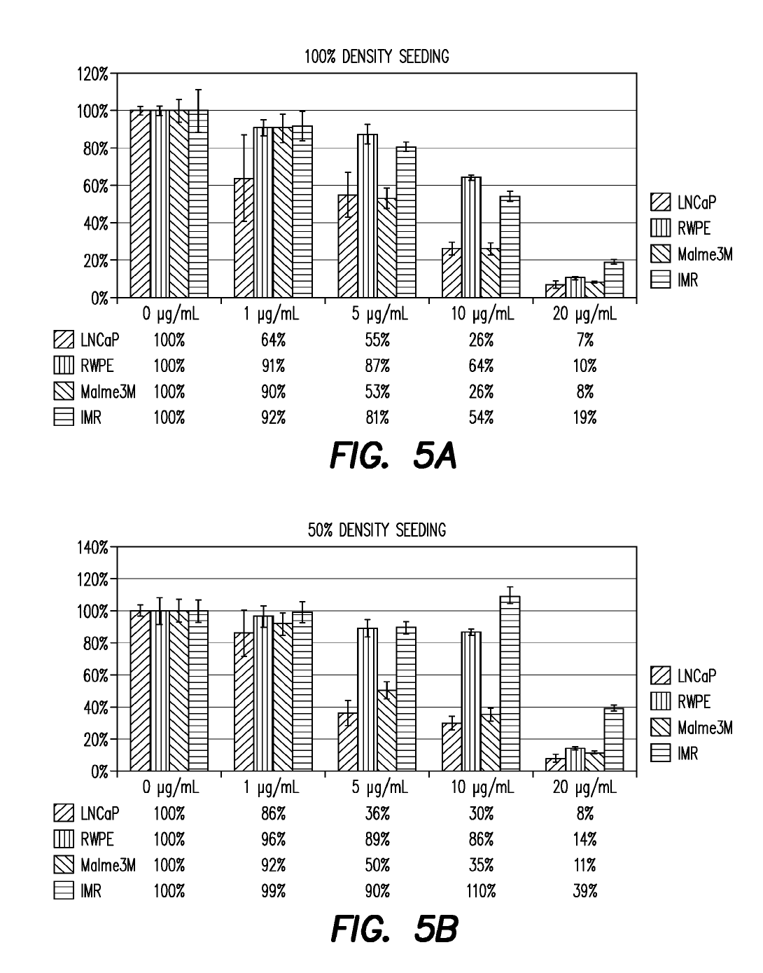

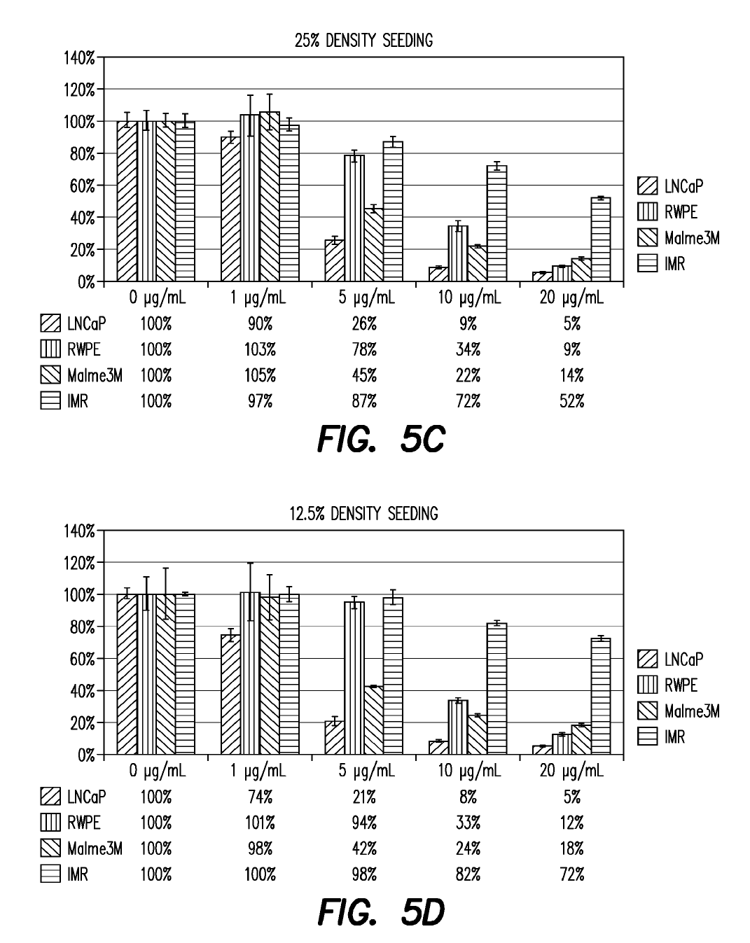

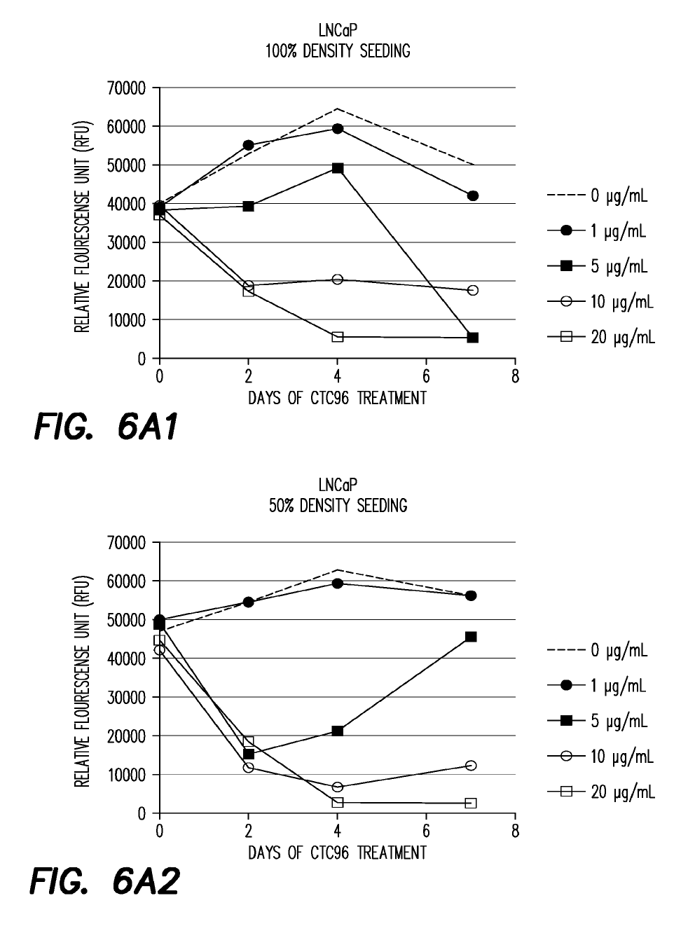

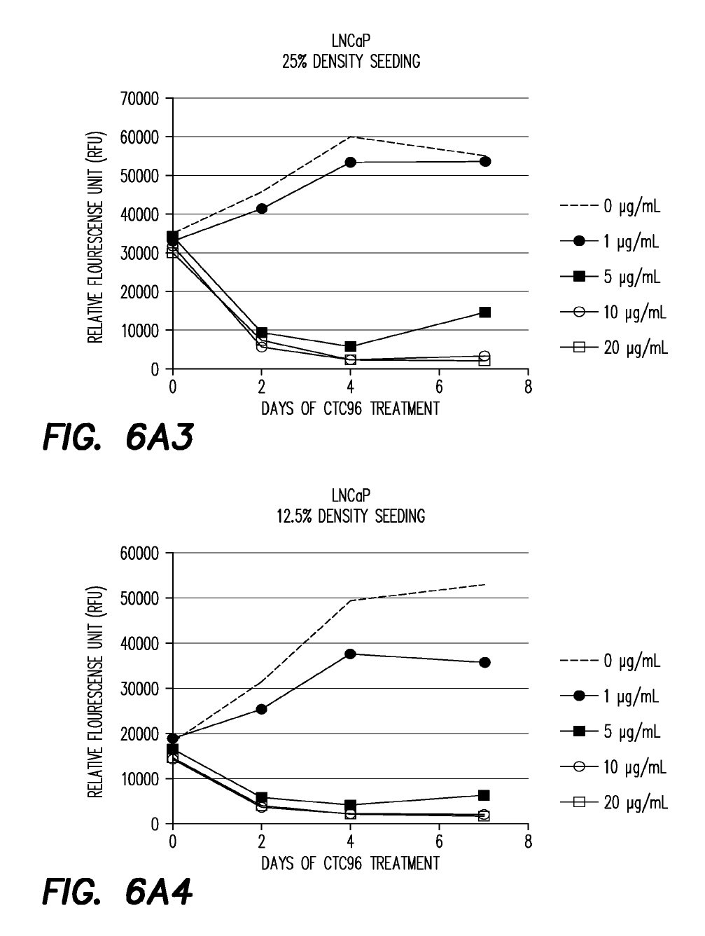

The cell growth and test media was RPM11640/2% FBS. All cell lines were acclimated in this low-serum condition for about two weeks prior to treatment with CTC-96. CTC-96 treatment was extended to 7 days from the 3 days previously used. Cell viability and proliferation was measured and medium was changed on day 0, 2, 4 and 7. Fresh CTC-96 added during every medium change. Incubation for cell viability and proliferation assay was performed for 2 hours. The results are shown in FIGS. 4 and 5.

### H Overall Proliferation of Cell Lines in RPMI1640/2% FBS

All cell lines were stagnant for the first two days before proliferation occurred. This was indicated by the relatively flat trendline from day 0 to day 2 in untreated control cells for all seeding densities and all cell lines. Significant cell loss was seen with LNCaP at 100% density seeding, due to partially peeling off of the monolayer. LNCaP cells are known to have weak binding to tissue culture vessels and ATCC recommends that cells should not be disturbed within 48 hours after passage. Our study confirmed this characteristic of LNCaP. The effect was more pronounced at high confluency, where cells sometimes came off in sheets.

All cell lines, except for 1MR, proliferated well: the growth curve showed a plateau in high density seeding, while keeping a straight upward slope in the low density seeding. The actual achieved seeding density for IMR was much lower than expected. Therefore, IMR cells did not proliferate much at low density seedings. See FIG. 4D3-4. Theoretically. it is possible that the IMR cell proliferation rate decreased after being maintained in a low-serum condition for a long period of time A new batch of IMR cells with shorter acclimation in low-serum condition was successfully utilized in the next confirmation study.

### I CTC-96 Demonstrated Anti-Cancer Efficacy Against Malignant Cell Lines with Gli-1 Up-Regulation

Although all normal and malignant cell lines showed a dose-dependent response to CTC-96, both malignant cell lines were more sensitive to the drug than their normal sister cell lines. LNCaP and Malme-3M showed almost no change, or even had fewer cells than originally seeded with 5 μg/mL of CTC-96 (see FIGS. 4A and C). At 10 μg/mL. CTC-96 also inhibited the proliferation of normal cell lines, RWPE and IMR, However, they still had a higher percentage of cells than LNCaP and, Malme-3M at the 5 μg/mL level (see FIG. 5).

### J A Confirmation Study Revealed Similar Anti-Cancer Efficacy for CTC-96

The study was performed as described in GP689 with modifications as follows:

A new batch of all four test cell lines were acclimated in RPMII 640/2% FBS about 10 days prior to CTC-96 treatment. CTC-96 treatment started after 2 days of adaptation. A 96-well plate was used at the at the start of proliferation in an attempt to eliminate the peeling of LNCaP monolayers. A manual multichannel pipette was utilized to change medium on day 0. Peeling still occurred in one plate. That plate was arbitrarily assigned the last time point day 7 in order to have good plates of LNCaP for the first three time points in a row.

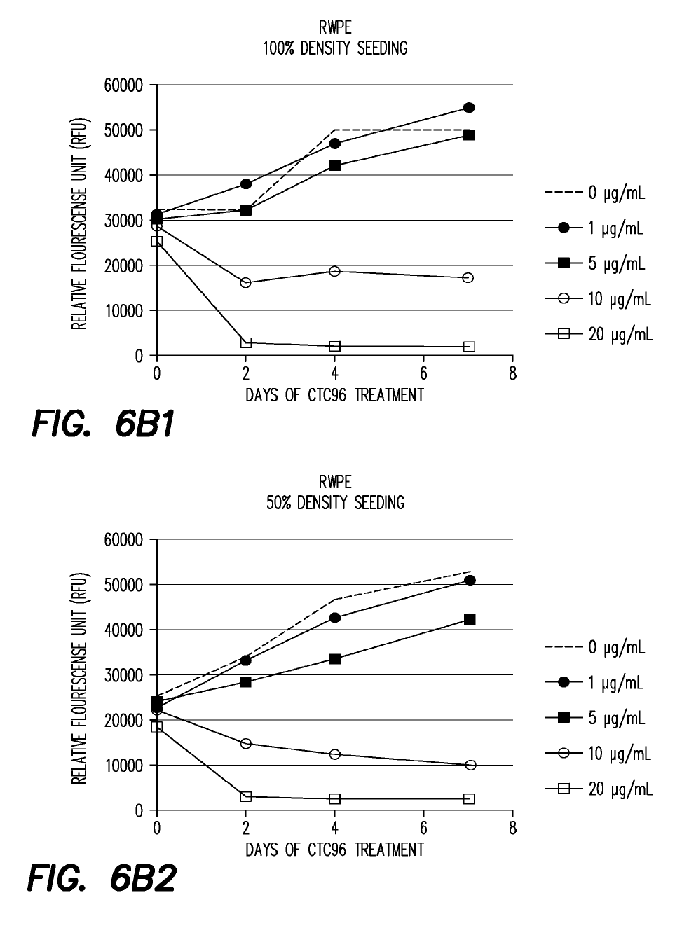

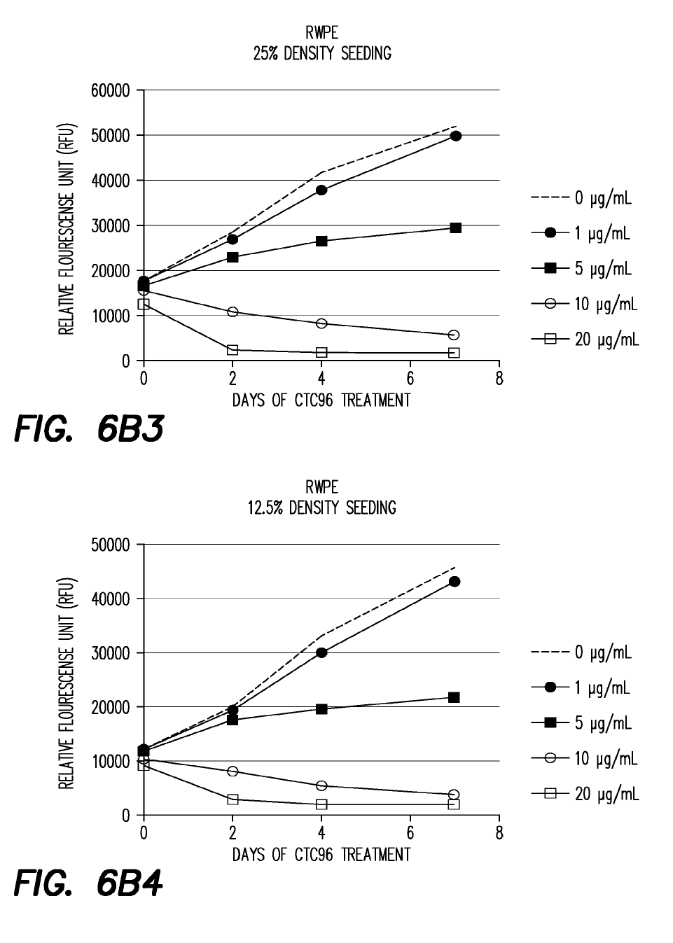

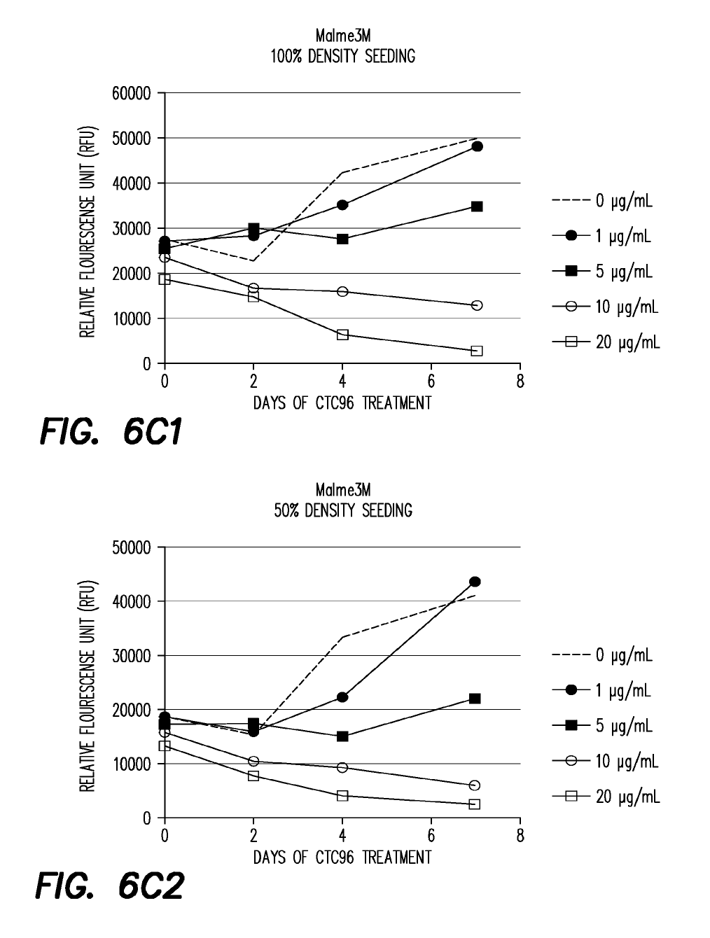

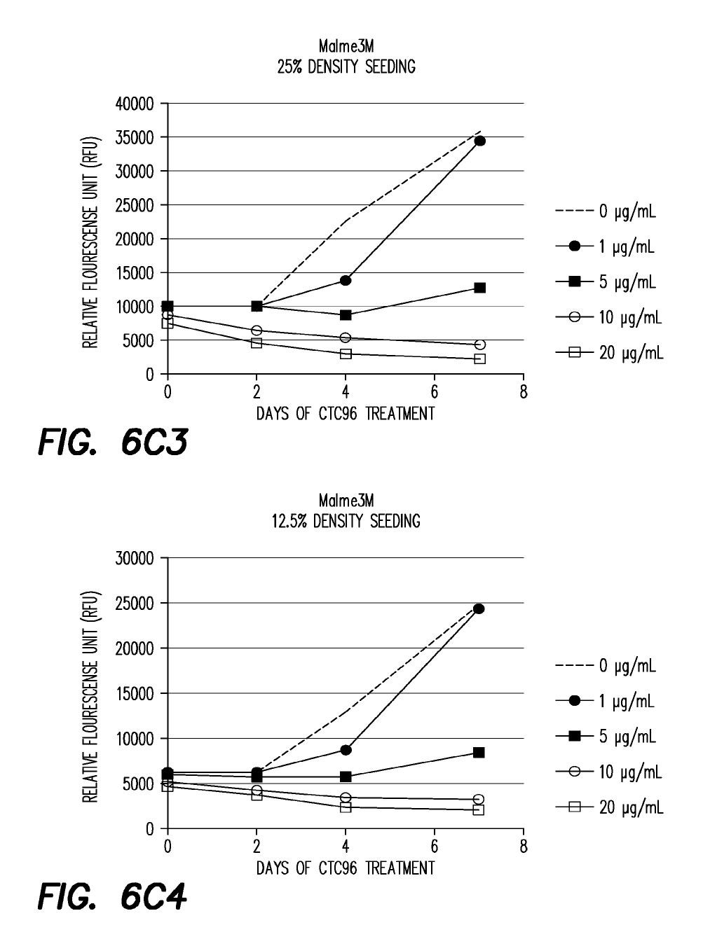

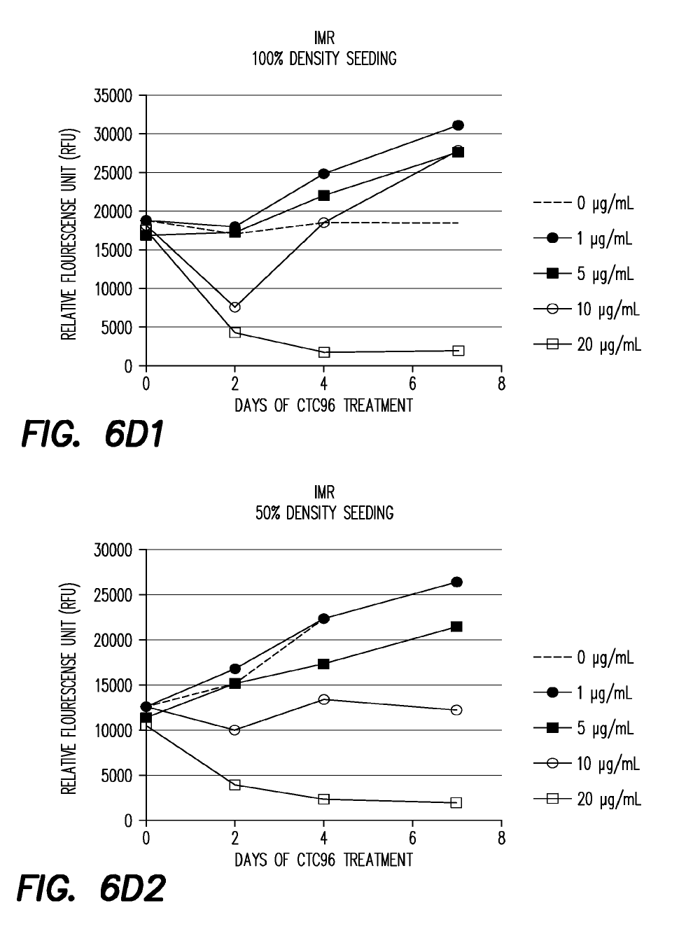

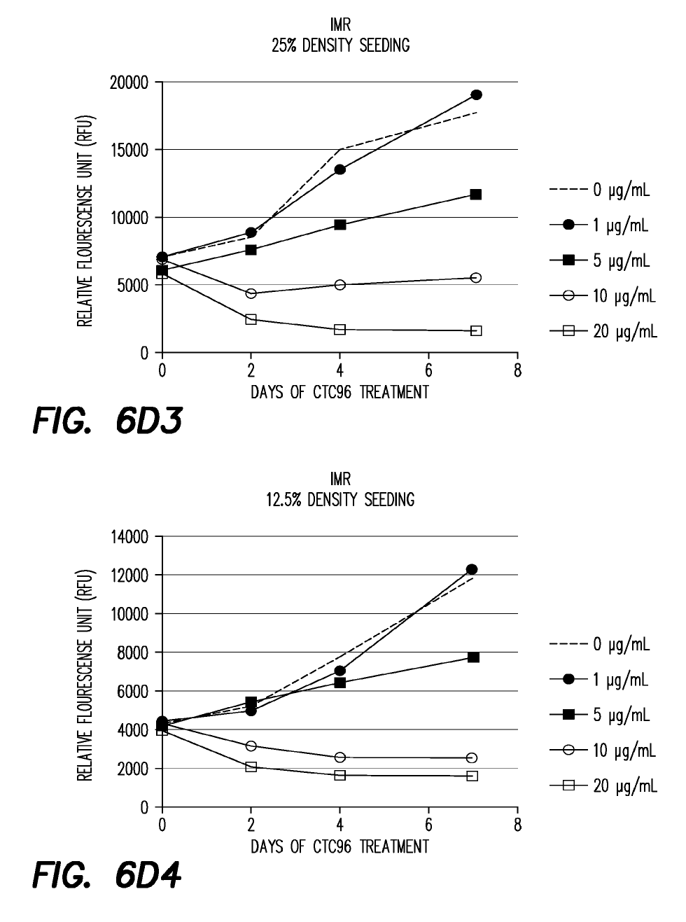

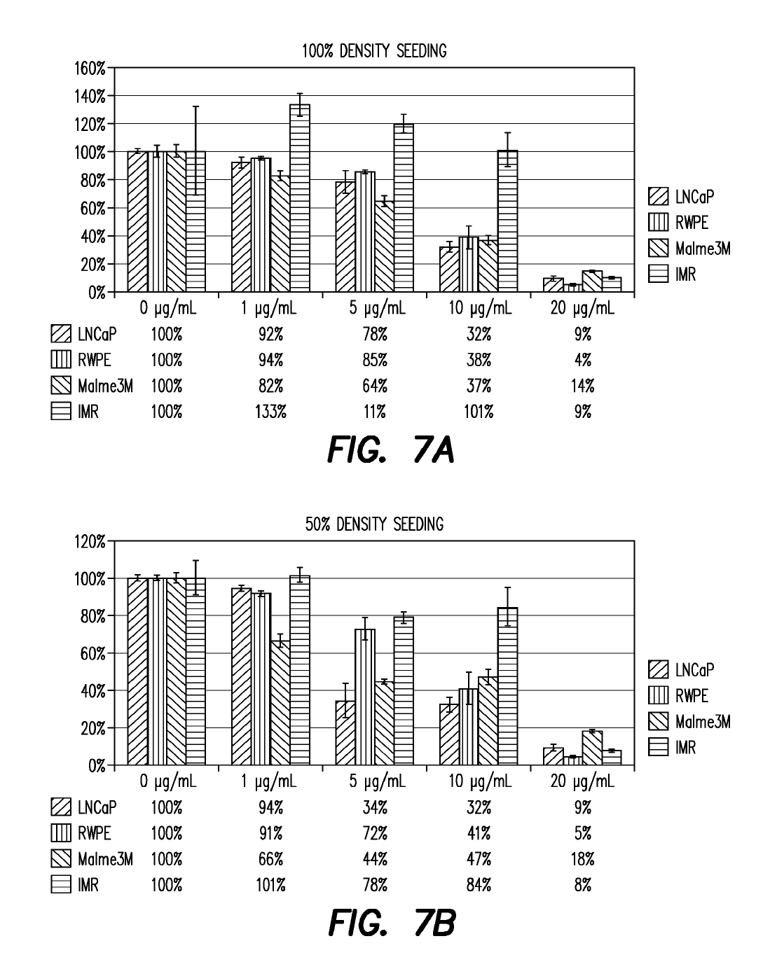

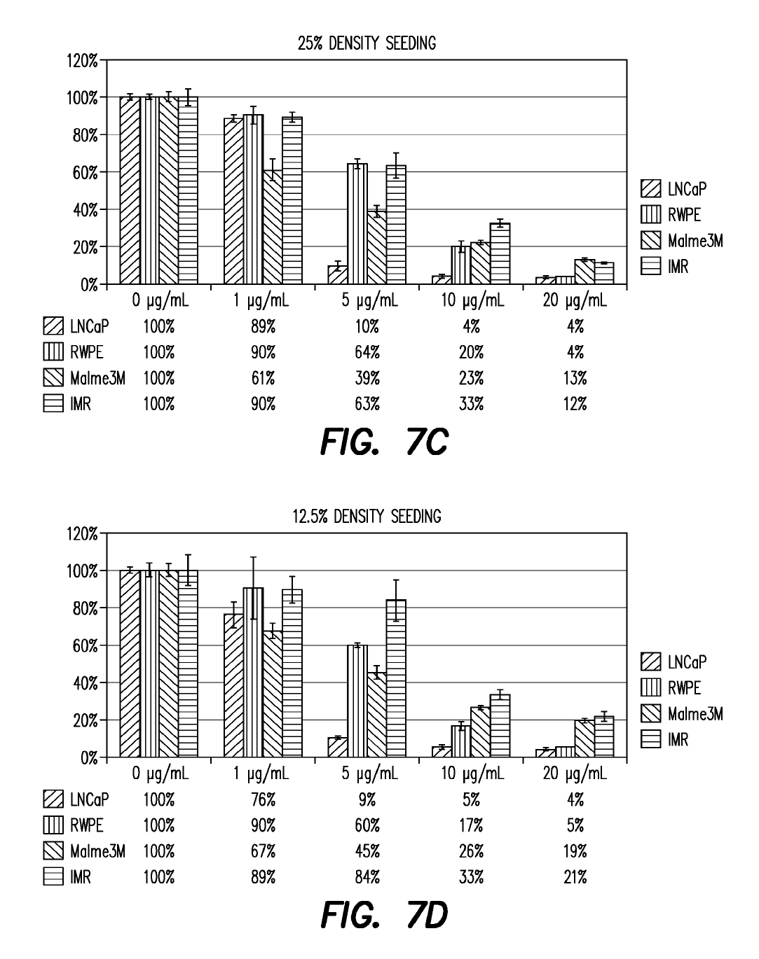

For medium changes on days 2, 4 and 7, only half of the media was replaced to completely eliminate the peeling in LNCaP. This method was utilized for the other three cell lines in order to keep the entire test system consistent for all the cell lines. The replacement media contained 1×CTC-96 concentrations and therefore only half of CTC-96 was freshly replaced. Incubation for cell viability and proliferation assay was performed for strictly 2 hours. Allover proliferation was good for all cell lines (see FIG. 6). Untreated ENCaP cells became 100% confluent in all seeding conditions on day 7. The other untreated three cell lines all demonstrated linear proliferation plots. 4.2.5.2.1.3. The day 4 RFU reading was chosen in the proliferation percentage analysis because the peeling off in LNCaP Day 7 plate led to decrease on RFU so much that the trendline of untreated cells turned downwards instead of plateauing in high density seeding, (see FIG. 6 Al 3). Decay of CTC-96 may occur over 7 days period and inhibition might be lowered towards the end of treatment. The upward turn of the trendline for malignant cell lines in 5 μg/mL may indicate this decay, (see FIG. 6 A2-3, CI-4). Malignant cell lines were more sensitive to CTC-96 than their sister normal cell lines. 5 μg/mL CT 96 showed greater inhibition on LNCaP and Malme-3M while 10 μg/mL produced a lower degree of inhibition in RWPE and IMR (see FIG. 7).

### K. Conclusion

Standardized culture conditions were established for all four cell lines. Two of these, LNCaP and Malme 3M, were derived from metastatic sites of prostate cancer and melanoma, respectively. Both of these cell lines showed distinct over-expression of Gli-1 transcription factor. The other two were non-malignant RWPE-1 derived from prostate epithelium and 1MR 90, a diploid cell line derived from male lung epithelium. The latter is a genuine normal cell line whereas RWPE-1 is an immortalized cell line by virtue of insertion of some HPV 18 DNA sequences. As described above, the cell lines were grown in RPMI 1640 medium containing 2% fetal bovine serum. Under these conditions, the cells of these lines showed proliferation (FIGS. 3 and 4). The effect of CTC 96 on cell proliferation and cell elimination by the drug are as follows:

Cell Proliferation.

The effect of CTC-96 on cell proliferation is best seen under the conditions: drug concentrations of 1, 5 μm/mL, with initial cell seeding densities of 50% and 25%. The results are shown in FIG. 4 and of the repeat experiment shown in FIG. 6.

At 50% cell density LNCaP (prostate) proliferation is diminished by about 30-40% by 5 μm/mL of CTC-96. By comparison, the proliferation of its non-malignant counterpart, RWPE-1, is minimally affected by the same drug concentration. At 25% cell density, proliferation completely ceases in the malignant cell line at 5 t μm/mL whereas RWPE-1 still proceeds proliferating, albeit, at a reduced rate. Malme 3M (melanoma) ceases proliferating at 1 μg/mL whereas IMR 90 (normal diploid) continues proliferating at 5 μg/mL

Cell Elimination (FIGS. 4 and 6).

At initial seeding densities of 50% and 25%, LNCaP cells treated with 5 μm/mL of CTC-96 showed a decrease of 30-50% in cell number, compared to un-treated controls. There was no cell loss in the non-malignant counterpart (RWPF-1) although this line still proliferated at the concentration that killed the malignant cell line. Malme 3M was not killed, but stopped proliferating at 5 μg/mL drug concentration.

I claim:

1. A method for treating a human subject having a tumor by administering to the subject an antitumor effective amount of CTC-96.

2. The method of claim 1 wherein the CTC-96 is administered intravenously, intraperitoneally, intramuscularly, orally or intranasally.

3. A composition for the treatment of a human subject having a tumor comprising an antitumor effective amount of CTC-96 in admixture with a pharmaceutically acceptable carrier.
